(19) United States
(12) Patent Application Publication
(10) Pub. No.: US 2025/0207104 A1
(43) Pub. Date: Jun. 26, 2025

(54) METHOD FOR PRODUCING INNER EAR ORGANOIDS USING RETINOIC ACID AND INNER EAR ORGANOIDS PRODUCED BY THE METHOD

(71) Applicant: AJOU UNIVERSITY INDUSTRY-ACADEMIC COOPERATION FOUNDATION, Suwon-si (KR)

(72) Inventors: Yun-Hoon Choung, Seoul (KR); Jeong Hun Jang, Seongnam-si (KR); Young sun Kim, Hwaseong-si (KR); Jungho Ha, Suwon-si (KR)

(73) Assignee: AJOU UNIVERSITY INDUSTRY-ACADEMIC COOPERATION FOUNDATION, Suwon-si (KR)

(21) Appl. No.: 18/982,276

(22) Filed: Dec. 16, 2024

(30) Foreign Application Priority Data
Dec. 14, 2023 (KR) 10-2023-0182416

(51) Int. Cl. C12N 5/071; C12N 5/00

(52) U.S. Cl. CPC C12N 5/0697; C12N 5/0018; C12N 2501/385; C12N 2502/081; C12N 2502/092; C12N 2506/02; C12N 2506/45; C12N 2513/00

(57) ABSTRACT

The present disclosure relates to a method for producing an inner ear organoid with improved differentiation efficiency of hair cells. The inventors of the present disclosure have confirmed that when retinoic acid is treated at the stage of formation of the lamina propria during the production of an inner ear organoid, the differentiation potential of hair cells is excellent. Therefore, it is expected that the inner ear organoid produced according to the production method of the present disclosure can be advantageously utilized in hearing loss disease modeling, drug screening and regenerative medicine.

### CROSS-REFERENCE TO RELATED APPLICATION(S)

This application claims priority to and the benefit of Korean Patent Application No. 10-2023-0182416, filed on Dec. 14, 2023, the disclosure of which is incorporated herein by reference in its entirety.

### INCORPORATION BY REFERENCE OF SEQUENCE LISTING

The instant application contains a Sequence Listing which has been filed electronically in xml format and is hereby incorporated by reference in its entirety. Said xml copy, created on Mar. 13, 2025, is named sequence listing as filed and is 29,255 bytes in size.

### TECHNICAL FIELD

The present disclosure provides a method for promoting differentiation of hair cells during inner ear organoid formation. Specifically, the present disclosure provides an inner ear organoid produced through treatment of retinoic acid at a stage of otic placode formation to promote differentiation of hair cells, thereby including a large amount of hair cells.

### RELATED ART

Hearing loss refers to a phenomenon in which sounds cannot be heard well due to abnormalities in the outer, middle or inner ear. The occurrence of hearing loss can be classified into aging, sudden, noise-induced, ototoxic and hereditary causes, and is mainly characterized by damage and death of auditory hair cells in the organ of Corti in the inner ear.

In hearing loss research, it is difficult to obtain cells derived from actual patients, and animal auditory cell lines and cochlea tissues cannot mimic the actual inner ear, which limits the development of drugs to prevent and treat hearing loss. Additionally, in a situation where human and experimental animal research is increasingly restricted due to ethical issues, organoids are a groundbreaking technology in that they reproduce actual organs using human and animal stem cells.

An organoid is a three-dimensional ‘organ-like body’ that similarly reproduces the body organs that serve as research models in vitro, and it has the great advantage of allowing high-speed new drug screening through organoid mass production technology, and allowing disease modeling and customized treatment screening with genetic information from actual patient tissues.

Inner ear organoids have been developed as a three-dimensional culture protocol since 2014, but the efficiency of organoid production is very low, and the number of hair cells in the produced organoids is very small, about 1% of the total cells, thereby making them unsuitable for drug screening.

The auditory hair cells in the organ of Corti, which are the auditory receptors inside the cochlea, are expressed in the sensory epithelium before the supporting cells, and the differentiation of hair cells and supporting cells is determined through lateral inhibition of the Notch signal. Gamma secretase (y-secretase) is a hydrolytic enzyme that cleaves the transmembrane region of Notch, which releases the Notch intracellular domain (NICD) into the cell. When NICD moves into the nucleus, Atoh1, which is a transcription factor related to hair cell differentiation, is suppressed, and genes such as Hes1 and Hes5 are expressed. Therefore, gamma secretase inhibitors can induce hair cells from supporting cells by reducing the expression of NICD.

Meanwhile, retinoic acid (RA) is a biologically active derivative of vitamin A and is known to play an important role in various developmental processes including embryogenesis and tissue differentiation. In addition, the role of retinoic acid (RA) signaling in patterning the frequency-tuned hair cell phenotype in the cochlea has been emphasized.

Accordingly, the inventors of the present disclosure completed the present disclosure by confirming that the number of hair cells in the inner ear organoid was efficiently increased by adding retinoic acid (RA) in order to maximize hair cell differentiation in the organoid production process similar to the differentiation process from non-sensory cells to hair cells in vivo.

### SUMMARY

The present disclosure has an object to provide a method for producing an inner ear organoid.

The present disclosure is directed to providing a composition for promoting differentiation of hair cells during inner ear organoid formation.

In addition, the present disclosure is directed to providing a treatment time and concentration of a composition for promoting differentiation of hair cells.

In addition, the present disclosure is directed to providing a method for screening an inner ear organoid including a large amount of hair cells and a composition for treating hearing loss using the same.

However, the technical problems to be achieved by the present disclosure are not limited to the problems mentioned above, and other problems that are not mentioned will be clearly understood by those skilled in the art from the description below.

Hereinafter, the present disclosure will be described in detail. The advantages and features of the present disclosure and the embodiments that achieve the same will be clearer with reference to the embodiments described below. However, the present disclosure is not limited to the embodiments disclosed below, but can be implemented in various different forms, and these embodiments are provided only to make the disclosure of the present disclosure complete and to fully inform those skilled in the art to which the present disclosure pertains of the scope of the invention, and the present disclosure is defined only by the scope of the claims. Like reference numerals refer to like elements throughout the specification.

Unless otherwise defined, all terms (including technical and scientific terms) used in the present specification may be used in meanings that can be commonly understood by those skilled in the art to which the present disclosure pertains. In addition, terms defined in commonly used dictionaries shall not be interpreted ideally or excessively unless explicitly specifically defined. The terms used in the present specification are intended to describe the embodiments and are not intended to limit the present disclosure. In the present specification, singular forms also include plural forms unless specifically stated in the phrase.

In order to achieve the above object, the present disclosure provides a composition for producing an inner ear organoid, including retinoic acid. The composition of the present disclosure is treated during the formation process of an inner ear organoid to promote differentiation of hair cells.

In the present specification, “organoid” is also called a pseudo-organ, and cells separated from stem cells or organs are re-aggregated/recombined through three-dimensional culture to achieve tissue formation similar to actual organs and reproduction of the functions of living organs such that it may be used as a model for various studies such as development and diseases.

In the present disclosure, the composition may be treated during the otic placode formation stage among the inner ear organoid production stages. Specifically, it is treated on day 10 to day 20 (preferably, day 12 to day 18, for about 6 days) from a start date of cell differentiation for inner ear organoid formation (after the formation of embryoid bodies (EBs), a start date of cell differentiation).

The composition of the present disclosure may include retinoic acid at a concentration of 1 μM to 20 μM, and most preferably include retinoic acid at a concentration of 1 μM to 5 μM. When the concentration of retinoic acid to be treated exceeds 20 μM, the number of hair cells formed may decrease.

In addition, the present disclosure a method for producing an inner ear organoid, including a) culturing embryonic stem cells or induced pluripotent stem cells to form a true endoderm; b) forming a non-neural ectoderm; c) forming an otic-epibranchial progenitor domain; d) forming an otic placode; and e) differentiating hair cells, supporting cells and neurons. The present disclosure may produce an inner ear organoid with a moted hair cell differentiation potential by treating a composition including retinoic acid in step d). Specifically, step d) is preferably performed on day 12 to day 18 after a start of cell differentiation for inner ear organoid formation (day 12 to day 18 from a start date of step a).

In addition, the present disclosure provides an inner ear organoid produced according to the above production method and including a large amount of hair cells. The inner ear organoid produced according to the present disclosure may be used for screening a drug for treating hearing loss.

When the composition including retinoic acid according to the present disclosure is treated in the process of producing an inner ear organoid, the differentiation of hair cells is promoted. Since the inner ear organoid produced according to the present disclosure includes a large amount of hair cells, it may be advantageously used in inner ear-related experiments such as screening for drugs for treating hearing loss.

### BRIEF DESCRIPTION OF THE DRAWINGS

FIG. 1 is a flow chart showing the culture process of an inner ear organoid according to the present disclosure. Referring to FIG. 1, retinoic acid (or a composition including the same) can be treated at a stage of otic vesicle formation s (more specifically, a stage of otic placode formation) among stages of inner ear organoid differentiation.

FIG. 2 is the results of observing the organoid morphology of human induced pluripotent stem cells (CMC-11 and mND2-0) produced according to the present disclosure under a microscope on day 20 (scale bar: 200 μm).

FIG. 3 is the results of confirming the expression of mRNA related to hair cell differentiation on day 25 of the production of the inner ear organoid according to the present disclosure through quantitative RT-PCR (qRT-PCR). A of FIG. 3 is the results according to the CMC-11 cell line, and B of FIG. 3 is the results according to the mND2-0 cell line.

FIG. 4 is the results of immunostaining each organoid with Myosin VIIA, phalloidin, TUJ1, SOX2, DAPI and the like on day 90 of inner ear organoid production using the mND2-0 cell line according to one embodiment of the present disclosure, and observing fluorescence with a confocal microscope. The upper part of FIG. 4 and the 1 μM retinoic acid treatment group (scale bar: 10 μm) are images confirming the shape of the organoid at high magnification, respectively, and in the middle and lower parts of FIG. 4, the 5 μM, 10 μM and 20 μM retinoic acid treatment groups (scale bar: 50 μm) are the results of observing the shapes of the inner ear organoids at low magnification, respectively.

FIG. 5 is the results of quantitative RT-PCR for the expression of mRNA related to hair cells in inner ear organoids 90 days after treatment with retinoic acid at various concentrations for 6 days from day 2 to day 18 according to the inner ear organoid production method of the present disclosure.

### DETAILED DESCRIPTION OF THE EMBODIMENTS

Hereinafter, in order to help understand the present disclosure, examples will be provided to explain in detail. However, the following examples are only intended to illustrate the contents of the present disclosure, and the scope of the present disclosure is not limited to the following examples. The examples of the present disclosure are provided to more completely explain the present disclosure to one of ordinary skill in the art.

As used herein, the term “hair cell” refers to a cell located in the Organ of Corti of the cochlea in the inner ear, and the term “differentiation potential” refers to the ability of cells to specialize in structure or function while they divide and proliferate and grow. The inner ear organoid produced according to the present disclosure may contain a large amount of hair cells because the differentiation potential of hair cells is improved.

As used herein, the term “stem cell” refers to a cell that has the ability to differentiate into various cells and self-proliferation ability through an appropriate environment and stimulation, and it may be an adult stem cell, an induced pluripotent stem cell or an embryonic stem cell, and preferably an embryonic stem cell or an induced pluripotent stem cell, but is not limited thereto.

As used herein, the term “differentiation” refers to a phenomenon in which the structure or function of a cell becomes specialized while the cell divides and proliferates and the entire organism grows. In other words, it refers to a process in which cells, tissues and the like of a living organism change into a suitable form and function to perform the role given to each.

In the present disclosure, the process of forming an inner ear organoid through stem cell culture may be performed by treating an appropriate growth factor known to those skilled in the art to which the present disclosure pertains.

Basically, the inner ear organoid formation process includes \textcircled{1} a stage of definitive ectoderm formation, \textcircled{2} a stage of non-neural ectoderm formation, \textcircled{3} a stage of preplacodal ectoderm, \textcircled{4} a stage of otic-epibranchial placode domain formation, \textcircled{5} a stage of otic vesicle formation, and \textcircled{6} a stage of hair cell, support cell and neuron differentiation in that order.

The basic inner ear organoid culture method of the present disclosure other than a stage of retinoic acid treatment is not particularly limited, and known techniques may be used.

The composition including retinoic acid of the present disclosure may be treated at a stage of otic placode induction stage during 5 the stage of otic vesicle formation to promote the differentiation of hair cells.

In the present specification, the “matrigel” used in the inner ear organoid culture refers to a protein complex (product name of BD Bioscience) extracted from sarcoma cells of EHS (Engelbreth-Holm-Swarm) mice. The matrigel includes an extracellular matrix (ECM) such as laminin, collagen and heparin sulfate proteoglycan, and growth factors such as fibroblast growth factor (FGF), epiderma growth factor (EFG), insulin-like growth factor (IGF), transforming growth factor-beta (TGF-β) and platelet-derived growth factor (PDGF).

As used herein, the term “FGF2” refers to the fibroblast growth factor 2 protein encoded by the FGF2 gene. Information on the protein sequences used in the present disclosure, such as FGF2, may be found through known databases (e.g., NCBI Reference Sequence: NP_001997, NP_001348594).

Hereinafter, the present disclosure will be described in more detail through examples. These examples are intended only to illustrate the present disclosure, and it will be apparent to those skilled in the art that the scope of the present disclosure is not to be construed as being limited by these examples.

### Example 1

### 1-1. Culture Preparation

Human induced pluripotent stem cells (hiPSC) were used to differentiate an inner ear organoid, and for the cell lines, CMC-hiPSC-011 (KNIH) provided by the National Stem Cell Bank of Korea and mND2-0 (WiCell) purchased from WiCell Research Institute were used. hiPSCs were cultured using mTeSR Plus (StemCell Technologies).

### 1-2. Culturing Inner Ear Organoids

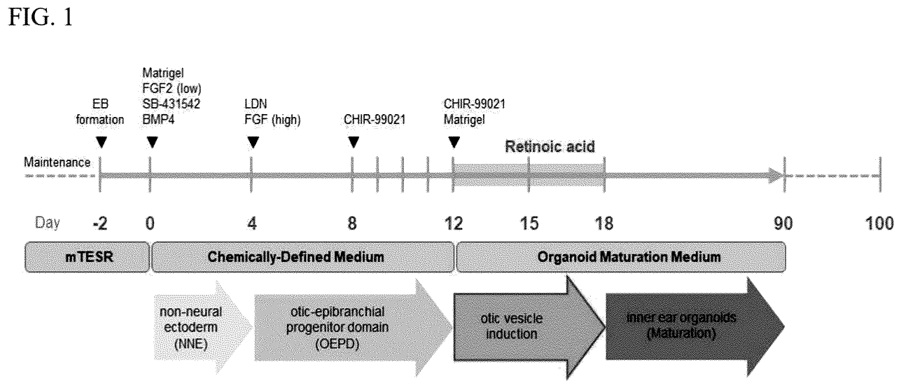

Human induced pluripotent stem cells (hiPSC; CMC-11, mND2-0) were differentiated into inner ear-like organoids for 90 days according to the method of the related art document [Koehler K R, Hashino E. 3d mouse embryonic stem cell culture for generating inner ear organoids. Nature Protocols 2014; 9:1229-44] according to Example 1-1. Specifically, cells in which embryoid bodies were formed after stem cell culture were differentiated in a matrigel medium including FGF2 (low), SB-431542 and BMP4. On day 3 of differentiation, 10 ng/ml of recombinant BMP4 (Stemgent, Beltsville, MD, USA) and 1 μM SB431542 (Stemgent) were added to the cultured cells to induce a non-neural ectoderm (a stage of non-neural ectoderm induction). On day 4, 25 ng/mL FGF2 (Peprotech, Rocky Hill, NJ, USA) and 1 μM LDN-193189 (Stemgent) were added to induce a preplacodal ectoderm (a stage of preplacodal ectoderm induction). Thereafter, the cells were cultured for 2 days, and on day 8, the medium was treated with CHIR-99021, and on day 12, the medium was replaced with 1% Matrigel medium including CHIR-99021 (a stage of otic vesicle induction). The control group was treated with CHIR-99021, and the experimental group was added with retinoic acid on day 12, and retinoic acid was treated in a maturation medium without Matrigel on day 15. On day 18, it was replaced with organoid maturation medium in the control and experimental groups were. Thereafter, the organoids were cultured in the organoid medium (a stage of second maturation). A schematic diagram of the differentiation process is shown in FIG. 1.

In summary, the experimental groups according to the present disclosure were treated with retinoic acid at concentrations of 1 μM, 5 μM, 10 μM and 20 μM for 6 days from day 12 of cellular differentiation, and the control group formed an organoid in the same manner as above, but was not separately treated with retinoic acid.

### Example 2

2-1. Confirmation of the Number of Organoid Buds Generated According to the Treatment with Composition Including Retinoic Acid

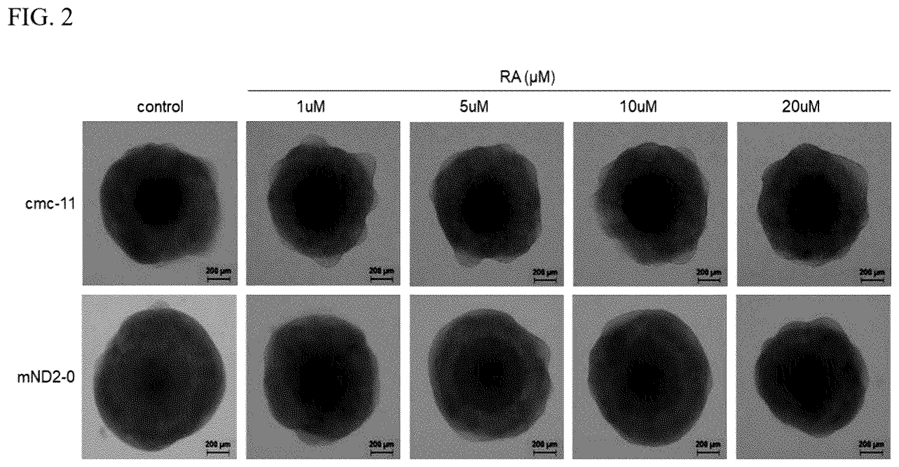

In this example, the organoid morphology of human induced pluripotent stem cells (CMC-11 and mND2-0) produced by the method of Example 1-2 was observed under a microscope on day 20 (see FIG. 2, scale bar: 200 μm). In the case of the organoids produced according to the present disclosure (experimental groups), it was confirmed that organoids were formed in a size and shape similar to the control group, regardless of the concentration of retinoic acid (RA) treated and regardless of the type of stem cell. Referring to FIG. 2, the number of organoid buds increased in the experimental groups compared to the control group.

2-2. Confirmation of mRNA Expression of Hair Cells Through Quantitative RT-PCR

In order to determine the degree of hair cell formation in the organoids produced according to Example 1-2, the expression of hair cell-related genes was confirmed using qRT-PCR on day 25 of organoid production. The control organoid formed in Example 1-2 was used as a qRT-PCR control (CTL) in this example.

Specifically, in order to perform qRT-PCR, total RNA was isolated from cultured organoids using RNAiso Plus ((TaKaRa, Shiga, Japan). The isolated total RNA was reverse transcribed into cDNA using PrimeScript 1st strand cDNA Synthesis Kit (TaKaRa). qRT-PCR experiments were performed using a CFX96 Real-Time PCR Cycler (Bio-Rad) and SYBR Green PCR Master Mix (NanoHelix Co., Ltd, Daejeon, South Korea) according to the manufacturer's instructions.

The primer sequences of each genetic marker used in this example are shown in Table 1 below.

TABLE 1

| | | | SEQ |
|---|---|---|---|
| | | | ID |
| Name of Gene | | Primer Sequence | NO: |
| DLX5 | Forward | TACCCAGCCAAAGCTTATGCCG | 1 |
| | Reverse | GCCATTCACCATTCTCACCTCG | 2 |
| OTX2 | Forward | GGAAGCACTGTTTGCCAAGACC | 3 |
| | Reverse | CTGTTGTTGGCGGCACTTAGCT | 4 |
| NR2F1 | Forward | TGCCTCAAAGCCATCGTGCTGT | 5 |
| | Reverse | CAGCAGCAGTTTGCCAAAACGG | 6 |
| FBXO2 | Forward | GCAGCAGTTCTACTTCCTGAGC | 7 |
| | Reverse | ACCATGCTCCACGTCACACCAG | 8 |
| NeuroD1 | Forward | GGTGCCTTGCTATTCTAAGACGC | 9 |
| | Reverse | GCAAAGCGTCTGAACGAAGGAG | 10 |
| ECAD | Forward | AGCGTGTGTGACTGTGAAGG | 11 |
| | Reverse | CTCTTCTCCGCCTCCTTCTT | 12 |
| PAX2 | Forward | GAGCGAGTTCTCCGGCAAC | 13 |
| | Reverse | GTCAGACGGGGACGATGTG | 14 |
| ATOH1 | Forward | GCAATGTTATCCCGTCGTTCAA | 15 |
| | Reverse | TCGGACAAGGCGTTGATGTA | 16 |
| ESPIN | Forward | CAGAGTGCAGGACAAAGACAA | 17 |
| | Reverse | GCAGCGTAGTGGATAGGCAG | 18 |
| MYO7A | Forward | GAGTCAGGCTTCCTCAGCTT | 19 |
| | Reverse | GTGACCAGGGCCACAATCTC | 20 |
| POU4F3 | Forward | CTGCAAGAACCCAAATTCTC | 21 |
| | Reverse | GGCTCTCATCAAAGCTTCCAAA | 22 |
| PRESTIN | Forward | ATGGCTACCAGGTTGACGGCAA | 23 |
| | Reverse | CCTCCTGAACAAGGCTTCGAGA | 24 |
| SOX2 | Forward | GCTACAGCATGATGCAGGACCA | 25 |
| | Reverse | TCTGCGAGCTGGTCATGGAGTT | 26 |

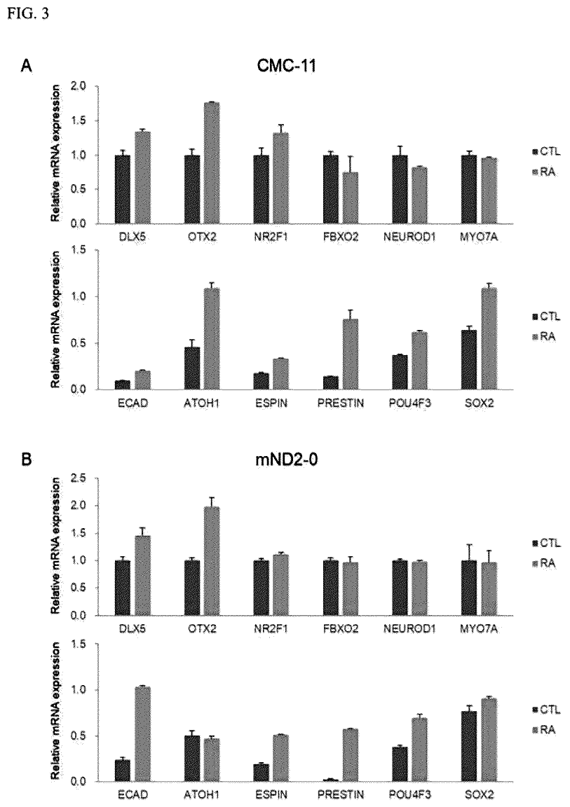

FIG. 3 is the results of quantitative RT-PCR (qRT-PCR) for confirming the expression of mRNA related to hair cell differentiation on day 25 of inner ear organoid production (day 25 of cell differentiation) according to this example. A of FIG. 3 is the results according to the CMC-11 cell line, and B of FIG. 3 is the results according to the mND2-0 cell line. Referring to FIG. 3, it can be confirmed that the expression levels of all markers increased in the experimental groups treated with retinoic acid on day 12 to day 18 of organoid production. In particular, the expression levels of hair cell markers and outer hair cell markers significantly increased in the retinoic acid (RA) treatment group, indicating that the inner ear organoids produced according to the present disclosure had promoted the differentiation of hair cells (Dorsal marker: DLX5, Ventral otic marker: OTX2, NR2F1, Otic marker: FBXO2, Neuronal marker: NeuroD1, Otocysts marker: ECAD, PAX2, Hair cell marker: ATOH1, ESPIN, MYO7A, POU4F3, Outer hair cell marker: PRESTIN, Supporting cell marker: SOX2).

### 2-3. Confirmation of Differentiation Potential of Hair Cells Using Immunostaining

In order to determine the expression of specific markers in the inner ear organoids produced according to Example 1-2, fluorescent immunostaining was performed on day 90 of organoid production (day 90 of cell differentiation).

Specifically, differentiated inner ear organoids were fixed with 4% paraformaldehyde for more than 24 hours and washed three times with phosphate-buffered saline (PBS). After washing with PBS, the aggregates were immersed in 15% and 30% (w/v) sucrose and embedded in Tissue-Tek OCT compound (Sakura Finetek, Flemingweg, Netherlands). Tissue blocks were cryo-sectioned into 10 μm sections, and the slides were blocked with 0.1% PBS-Tween (PBST) including 1% (w/v) bovine serum albumin (BSA) so as to prevent nonspecific binding of antibodies. Antibodies such as Myosin VIIA, which is a hair cell marker, Phalloidin, TUJ1, which is a neuron marker. and SOX2, which is a stem cell marker, were each treated at a concentration of 1:100 and maintained at 4° C. for more than 24 hours, then washed and treated with Hochest for nuclear staining, and fluorescence was observed using a confocal microscope.

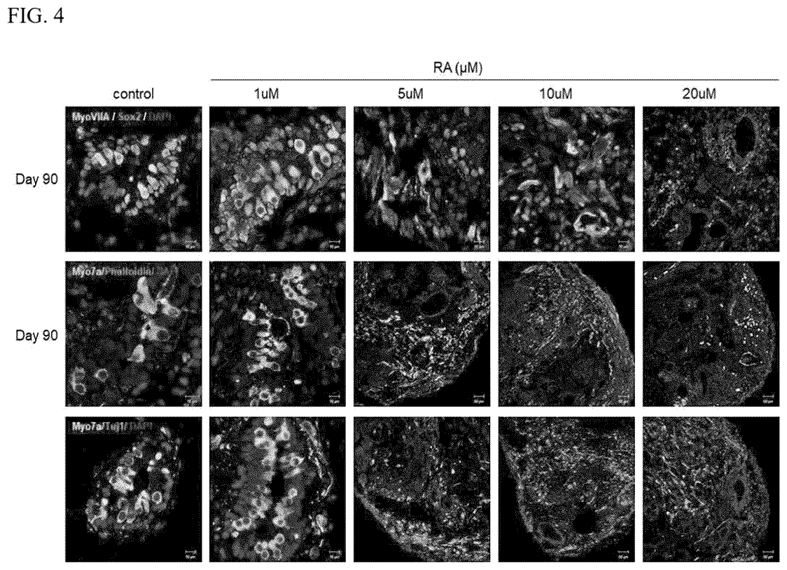

FIG. 4 is the results of staining organoids formed with the mND2-0 cell line according to this example with Myosin VIIA, Phalloidin, TUJ1, which is a neuron marker, SOX2, which is a stem cell marker, and DAPI, and observing fluorescence using a confocal microscope. Referring to FIG. 4, it can be confirmed that the expression of Myosin VIIA and the like increased in the retinoic acid treatment group (particularly, the retinoic acid treatment group at a concentration of 1 μM to 5 μM) compared to the control group, indicating that more retinoic cells were formed.

2-4. Confirmation of mRNA Expression of Hair Cells According to Retinoic Acid Treatment Concentration

In order to determine the degree of hair cell formation in the organoids produced according to Example 1-2, the expression of genes related to hair cells was confirmed using qRT-PCR on day 90 of organoid production (day 90 of cell differentiation). The control organoid formed in Example 1-2 was used as a qRT-PCR control (CTL) in this example.

Specifically, in order to perform qRT-PCR, total RNA was isolated from cultured organoids using RNAiso Plus ((TaKaRa, Shiga, Japan). The isolated total RNA was reverse transcribed into cDNA using PrimeScript 1st strand cDNA Synthesis Kit (TaKaRa). qRT-PCR experiments were performed using a CFX96 Real-Time PCR Cycler (Bio-Rad) and SYBR Green PCR Master Mix (NanoHelix Co., Ltd, Daejeon, South Korea) according to the manufacturer's instructions. The primer sequences of each genetic marker used in this example are shown in Table 2 below.

TABLE 2

| | | | SEQ |
|---|---|---|---|
| | | | ID |
| Name of Gene | | Primer Sequence | NO: |
| Myo VIIA | Forward | GAGTCAGGCTTCCT | 27 |
| | | CAGCTT | |
| | Reverse | GTGACCAGGGCCAC | 28 |
| | | AATCTC | |
| Atoh1 | Forward | GCAATGTTATCCCG | 29 |
| | | TCGTTCAA | |
| | Reverse | GCAATGTTATCCCG | 30 |
| | | TCGTTCAA | |
| Parvalbumin | Forward | CTGATGGCTGCTGG | 31 |
| | | AGACAAAG | |
| | Reverse | GAGATTGGGTGTTC | 32 |
| | | AGGGCAGA | |

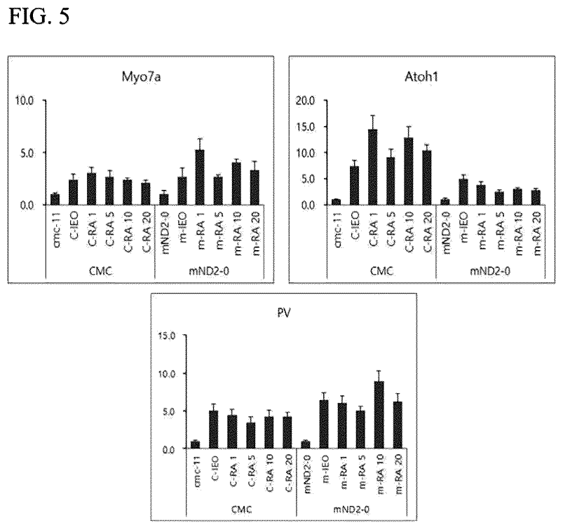

Referring to FIG. 5, as a result of the qRT-PCR of the inner ear organoids formed 90 days after retinoic acid treatment from day 12 to day 18 of organoid production (day 12 to day 18 of cell differentiation), it showed that the expressions of hair cell markers (MyoVIIA, Atoh1 and Parvalbumin (PV)) in two cell lines (CMC-11 and mND2-0) were higher in the retinoic acid treatment group (*P<0.05). That is, when retinoic acid is treated on day 12 to day 18, which is identified as the stage of otic placode formation during the process of inner ear organoid formation, it can be confirmed that hair cell differentiation is promoted, thereby forming inner ear organoids including a large number of hair cells.

The above description of the present disclosure is for illustrative purposes, and those skilled in the art will understand that it can be easily modified into other specific forms without changing the technical idea or essential features of the present disclosure. Therefore, it should be understood that the embodiments described above are exemplary in all respects and not limiting.

What is claimed is:

1. A composition for producing an inner ear organoid, comprising retinoic acid.

2. The composition of claim 1, wherein the composition is treated at a stage of otic placode formation during stages of inner ear organoid formation.

3. The composition of claim 1, wherein the composition comprises retinoic acid at a concentration of 1 μM to 5 μM.

4. The composition of claim 1, wherein the composition is treated on day 12 to 18 from a start date of cell differentiation for inner ear organoid formation.

5. The composition of claim 1, wherein the composition promotes differentiation of hair cells of the inner ear organoid.

6. A method for producing an inner ear organoid with a promoted differentiation potential of hair cells, the method comprising:
a) culturing embryonic stem cells or induced pluripotent stem cells to form a true endoderm;
b) forming a non-neural ectoderm;
c) forming an otic-epibranchial progenitor domain;
d) forming an otic placode; and
e) differentiating hair cells, supporting cells and neurons,
wherein a composition comprising retinoic acid is treated in step d).

7. The method of claim 6, wherein step d) is performed on day 12 to 18 after a start of cell differentiation for inner ear organoid formation.

8. The method of claim 6, wherein the composition treated in step d) comprises retinoic acid at a concentration of 1 μM to 5 μM.

9. An inner ear organoid, produced according to the method of claim 6.

10. The inner ear organoid of claim 9, wherein the inner ear organoid comprises a large amount of hair cells.


SEQUENCE LISTING (32 sequences)

SEQ ID NO: 1 - DNA, 22 bp, synthetic construct
  source 1..22: mol_type = other DNA
tacccagccaaagcttatgccg

SEQ ID NO: 2 - DNA, 22 bp, synthetic construct
  source 1..22: mol_type = other DNA
gccattcaccattctcacctcg

SEQ ID NO: 3 - DNA, 22 bp, synthetic construct
  source 1..22: mol_type = other DNA
ggaagcactgtttgccaagacc

SEQ ID NO: 4 - DNA, 22 bp, synthetic construct
  source 1..22: mol_type = other DNA
ctgttgttggcggcacttagct

SEQ ID NO: 5 - DNA, 22 bp, synthetic construct
  source 1..22: mol_type = other DNA
tgcctcaaagccatcgtgctgt

SEQ ID NO: 6 - DNA, 22 bp, synthetic construct
  source 1..22: mol_type = other DNA
cagcagcagtttgccaaaacgg

SEQ ID NO: 7 - DNA, 22 bp, synthetic construct
  source 1..22: mol_type = other DNA
gcagcagttctacttcctgagc

SEQ ID NO: 8 - DNA, 22 bp, synthetic construct
  source 1..22: mol_type = other DNA
accatgctccacgtcacaccag

SEQ ID NO: 9 - DNA, 23 bp, synthetic construct
  source 1..23: mol_type = other DNA
ggtgccttgctattctaagacgc

SEQ ID NO: 10 - DNA, 22 bp, synthetic construct
  source 1..22: mol_type = other DNA
gcaaagcgtctgaacgaaggag

SEQ ID NO: 11 - DNA, 20 bp, synthetic construct
  source 1..20: mol_type = other DNA
agcgtgtgtgactgtgaagg

SEQ ID NO: 12 - DNA, 20 bp, synthetic construct
  source 1..20: mol_type = other DNA
ctcttctccgcctccttctt

SEQ ID NO: 13 - DNA, 19 bp, synthetic construct
  source 1..19: mol_type = other DNA
gagcgagttctccggcaac

SEQ ID NO: 14 - DNA, 19 bp, synthetic construct
  source 1..19: mol_type = other DNA
gtcagacggggacgatgtg

SEQ ID NO: 15 - DNA, 22 bp, synthetic construct
  source 1..22: mol_type = other DNA
gcaatgttatcccgtcgttcaa

SEQ ID NO: 16 - DNA, 20 bp, synthetic construct
  source 1..20: mol_type = other DNA
tcggacaaggcgttgatgta

SEQ ID NO: 17 - DNA, 21 bp, synthetic construct
  source 1..21: mol_type = other DNA
cagagtgcaggacaaagacaa

SEQ ID NO: 18 - DNA, 20 bp, synthetic construct
  source 1..20: mol_type = other DNA
gcagcgtagtggataggcag

SEQ ID NO: 19 - DNA, 20 bp, synthetic construct
  source 1..20: mol_type = other DNA
gagtcaggcttcctcagctt

SEQ ID NO: 20 - DNA, 20 bp, synthetic construct
  source 1..20: mol_type = other DNA
gtgaccagggccacaatctc

SEQ ID NO: 21 - DNA, 20 bp, synthetic construct
  source 1..20: mol_type = other DNA
ctgcaagaacccaaattctc

SEQ ID NO: 22 - DNA, 22 bp, synthetic construct
  source 1..22: mol_type = other DNA
ggctctcatcaaagcttccaaa

SEQ ID NO: 23 - DNA, 22 bp, synthetic construct
  source 1..22: mol_type = other DNA
atggctaccaggttgacggcaa

SEQ ID NO: 24 - DNA, 22 bp, synthetic construct
  source 1..22: mol_type = other DNA
cctcctgaacaaggcttcgaga

SEQ ID NO: 25 - DNA, 22 bp, synthetic construct
  source 1..22: mol_type = other DNA
gctacagcatgatgcaggacca

SEQ ID NO: 26 - DNA, 22 bp, synthetic construct
  source 1..22: mol_type = other DNA
tctgcgagctggtcatggagtt

SEQ ID NO: 27 - DNA, 20 bp, synthetic construct
  source 1..20: mol_type = other DNA
gagtcaggcttcctcagctt

SEQ ID NO: 28 - DNA, 20 bp, synthetic construct
  source 1..20: mol_type = other DNA
gtgaccagggccacaatctc

SEQ ID NO: 29 - DNA, 22 bp, synthetic construct
  source 1..22: mol_type = other DNA
gcaatgttatcccgtcgttcaa

SEQ ID NO: 30 - DNA, 20 bp, synthetic construct
  source 1..20: mol_type = other DNA
tcggacaaggcgttgatgta

SEQ ID NO: 31 - DNA, 22 bp, synthetic construct
  source 1..22: mol_type = other DNA
ctgatggctgctggagacaaag

SEQ ID NO: 32 - DNA, 22 bp, synthetic construct
  source 1..22: mol_type = other DNA
gagattgggtgttcagggcaga